(19) United States
(12) Patent Application Publication
(10) Pub. No.: US 2010/0155279 A1
(43) Pub. Date: Jun. 24, 2010

(54) Container constructed substantially from card

(75) Inventor: Nicholas Berndt Wood, Sheffield (GB)

Correspondence Address: JAMES C. WRAY, 1493 CHAIN BRIDGE ROAD, SUITE 300, MCLEAN, VA, 22101, US

(73) Assignee: Econix Limited

(21) Appl. No.: 12/317,423

(22) Filed: Dec. 23, 2008

(51) Int. Cl. A61B 19/02; B65D 5/26

(52) U.S. Cl. 206/363; 229/126

(57) ABSTRACT

A container constructed substantially from card, comprising: a storage portion having a base and defining an opening through which items are insertable into the storage portion, and a closure portion foldable between a first configuration that allows access to the opening of the storage portion and a second configuration that closes the opening of the storage portion; and the closure portion comprises a flap having a free edge, the flap presenting a first securing element for securing the closure portion in the second configuration; wherein the flap further comprises a second securing element for securing the closure portion in the second configuration, and one of the first securing element and the second securing element is manually separable from the flap.

### BACKGROUND OF THE INVENTION

1. Field of the Invention

The present invention relates to a container constructed substantially from card and a method of use of a container.

2. Description of the Related Art

A container for storing biologically contaminated waste is described in British patent 2 421 495 in which the container is specifically configured to unseal during decontamination so as to assist the decontamination process. The container for storing biologically contaminated waste is also disclosed in British patent 2 421 498 (U.S. patent application Ser. No. 11/020,039) in which the container is specifically configured to facilitate stability when stored on flat surfaces. A container for a biologically contaminated waste is also disclosed in British patent number 2 444 719 in which an alternative type of seal is provided so as to facilitate temporary fastenings while the container is in use.

A problem with the aforesaid containers is that use is made of the containers during the dispersal process but problems arise in terms of the actual transportation of the containers for use on site.

### BRIEF SUMMARY OF THE INVENTION

According to an aspect of the present invention, there is provided a container constructed substantially from card, comprising a storage portion having a base and defining an opening through which items are insertable into said storage portion, and a closure portion foldable between a first configuration that allows access to said opening of said storage portion and a second configuration that closes said opening of said storage portion; and said closure portion comprises a flap having a free edge, said flap presenting a first securing element for securing said closure portion in said second configuration; wherein said flap further comprises a second securing element for securing said closure portion in said second configuration, and one of said first securing element and said second securing element is manually separable from said flap.

In a preferred embodiment, one of said first securing element and said second securing element that is manually separable from said flap is proximate to said free edge of said flap.

Preferably, there is a method of use for the aforesaid container comprising the steps of receiving said container with said closure portion secured in said second configuration by use of said second securing element, manually separating said second securing element from said flap, and securing said closure portion in said second configuration by use of said first securing element.

### BRIEF DESCRIPTION OF THE SEVERAL VIEWS OF THE DRAWINGS

FIG. 1 shows a container constructed from card;

FIG. 2 shows a first step in reconfiguring a closure portion;

FIG. 3 shows a second step with lips in a fully contacting position;

FIG. 4 shows a third step in which a flap and a lip assembly is then folded onto a top surface;

FIG. 5 shows a fourth step in which the lip assembly has been folded onto the surface of the container;

FIG. 6 shows the container of FIG. 1 in a sealed configuration;

FIG. 7 shows a first step for the use of the container identified in FIG. 1;

FIG. 8 illustrates a wraparound sleeve element that is configured to extend around the container of FIG. 1;

FIG. 9 shows the container of FIG. 1 in a closed configuration with its wraparound sleeve;

FIG. 10 shows the reception of a container with a wraparound sleeve;

FIG. 11 illustrates the tearing of a release element;

FIG. 12 shows the manual opening of the container after a the release element has been fully removed;

FIG. 13 illustrates the removal of an item from a container;

FIG. 14 illustrates the reuse of the container;

FIG. 15 shows the resealing of the container;

FIG. 16 illustrates method steps for the use of the container;

FIG. 17 illustrates further method steps;

FIG. 18 illustrates an embodiment in which a container is provided with a third temporary sealing element;

FIG. 19 illustrates a flap having an alternative configuration; and

FIG. 20 shows a further optional feature in the form of a handle for the container.

### DETAILED DESCRIPTION OF THE PREFERRED EMBODIMENTS

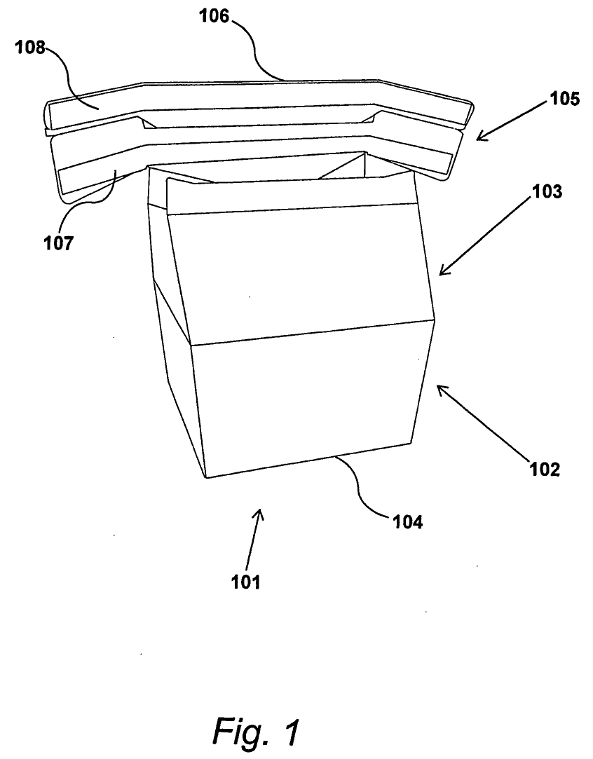

### FIG. 1

A container 101 constructed substantially from card is shown in FIG. 1. Container 101 comprises a storage portion 102 and a closure portion 103. The storage portion comprises a base 104 and defines an opening through which items are insertable into storage portion 102. Closure portion 103 is foldable between a first configuration that allows access to the opening of storage portion 102 and a second configuration that closes the opening of storage portion 102. Closure portion 103 comprises a flap 105 having a free edge 106. Flap 105 presents a first securing element 107 for securing closure portion 103 in the second configuration in which the storage portion is closed. Flap 105 further presents a second securing element 108 for securing closure portion 103 in the second configuration. As will be described in further detail below, one of the first and second securing elements 107, 108 is manually separable from flap 105. In accordance with this illustrated embodiment, the securing element that is manually separable from the flap is proximate to free edge 106 of flap 105. Thus, the second securing element 108 of container 101, which is proximate to free edge 106 of flap 105 is manually separable from flap 105.

In an embodiment, container 101 is constructed substantially from card having a thickness of approximately 0.5 mm. In an embodiment, the card is coated with a plastics material, such as polyethylene, providing a liquid-tight container. Such a container is then suitable for use for storing moist items.

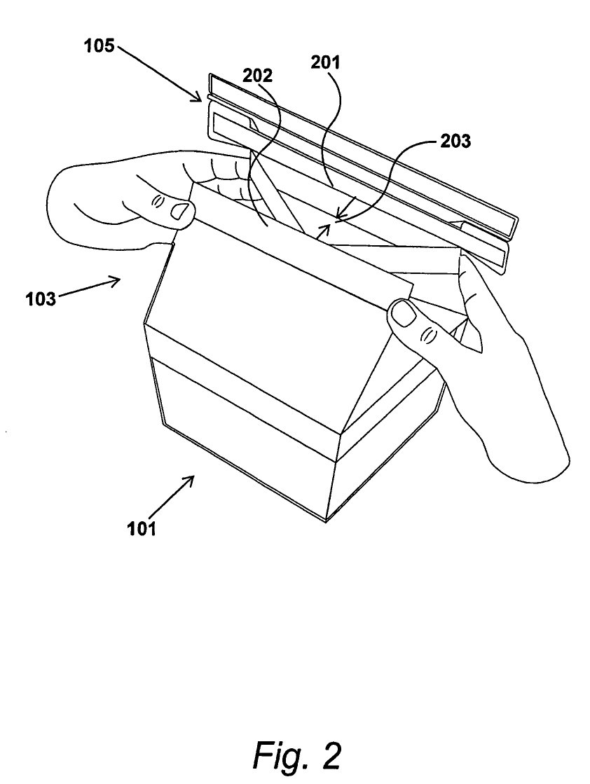

### FIG. 2

FIGS. 2 to 6 illustrate steps in reconfiguring closure portion 103 of container 101 from the first (open) configuration into the second (closed) configuration. Closure portion 103 comprises a first lip 201 and a second lip 202, which is opposite the first lip 201. Flap 105 extends from first lip 201. As shown in FIG. 2, lips 201 and 202 are configured to be manually brought together, as indicated by arrow 203.

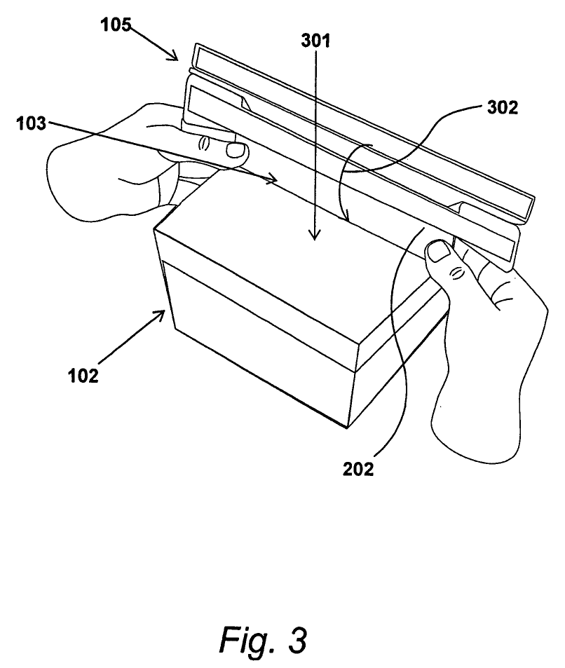

### FIG. 3

FIG. 3 shows the first lip (not shown in this Figure) and second lip 202 of closure portion 103 in the fully contacting position. The action of bringing the lips of closure portion 103 fully together, to form a lip assembly, results in the opening of storage portion 102 becoming closed, and the container presenting an upper surface 301. Flap 105 may now be folded over the pair of lips, as indicated by arrow 302.

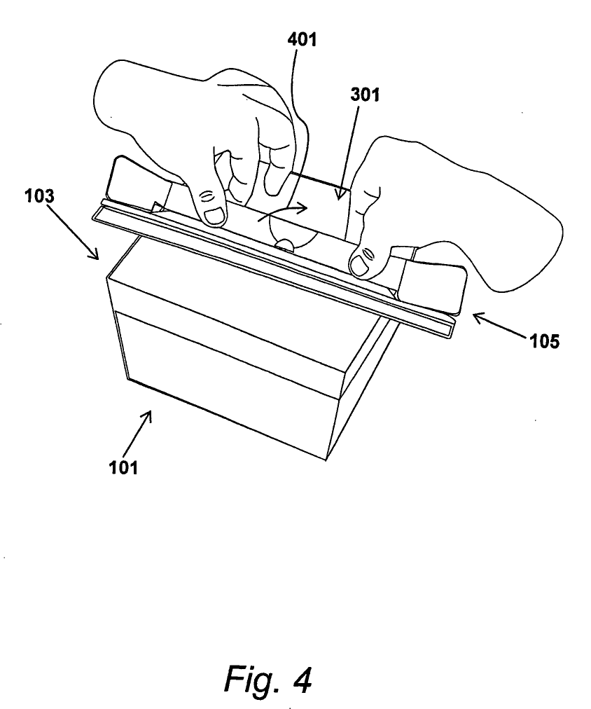

### FIG. 4

Following the folding of flap 105 over the lips (not shown in this Figure) of closure portion 103, flap 105 and the lip assembly is then foldable onto top surface 301 of container 101, as indicated by arrow 401.

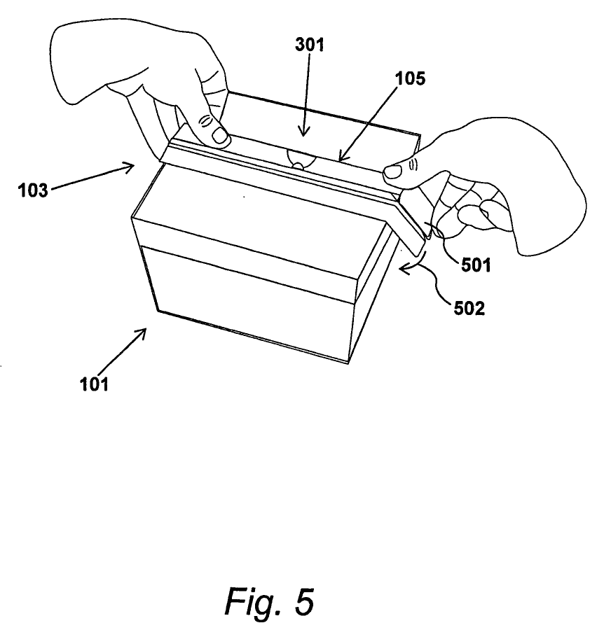

### FIG. 5

As shown in FIG. 5, one flap 105 and the lip assembly of closure portion 103 have been folded onto upper surface 301 of container 101, an overhanging portion at each end of flap 105, for example overhanging portion 501 may then be folded to lie against the side walling of container 101, as indicated by arrow 502.

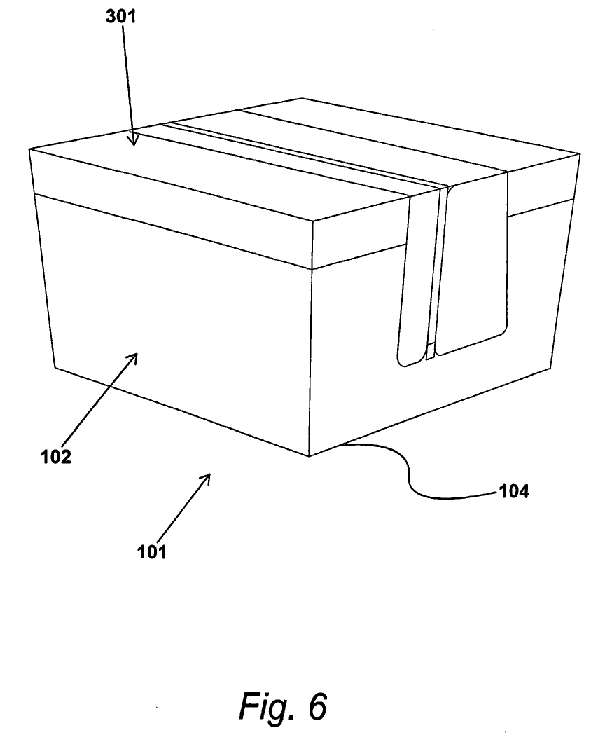

### FIG. 6

FIG. 6 shows container 101 in a sealed configuration. According to this illustrated embodiment, base 104 has a substantially square shape. Storage portion 102 comprises side walls that taper outwardly from the base. This arrangement facilitates stacking of a plurality of the containers when the containers are open. The stacking ability facilitates both storage and transportation. The closure portion is configured such that the container presents a substantially planar top surface 301 when the closure portion is in the second configuration. Again, this feature facilitates stacking of a plurality of the containers when the containers are closed to make storing and transporting more convenient.

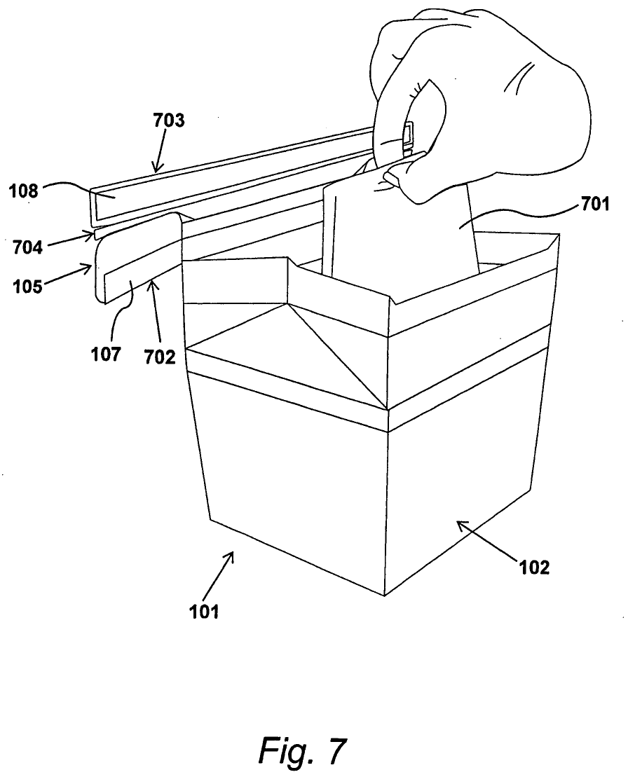

### FIG. 7

FIG. 7 to 15 illustrate a method of use of container 101. With container 101 open, an item 701 is placed inside closure portion 102. The first securing element 107 is presented by a first securing portion that is in the from of a strip that extends along the flap 105. Similarly, the second securing element 108 is presented by a second securing portion that is in the form of a strip that extends along flap 105. In an embodiment, the first and second securing element 107, 108 each comprise an adhesive surface. A peel-off strip may be provided over the adhesive surface. According to this illustrated embodiment, flap 105 comprises a release portion 704 that is in the form of a tear-off strip that extends along the flap between the first securing portion 702 and the second securing portion 703 and is manually separable from the flap.

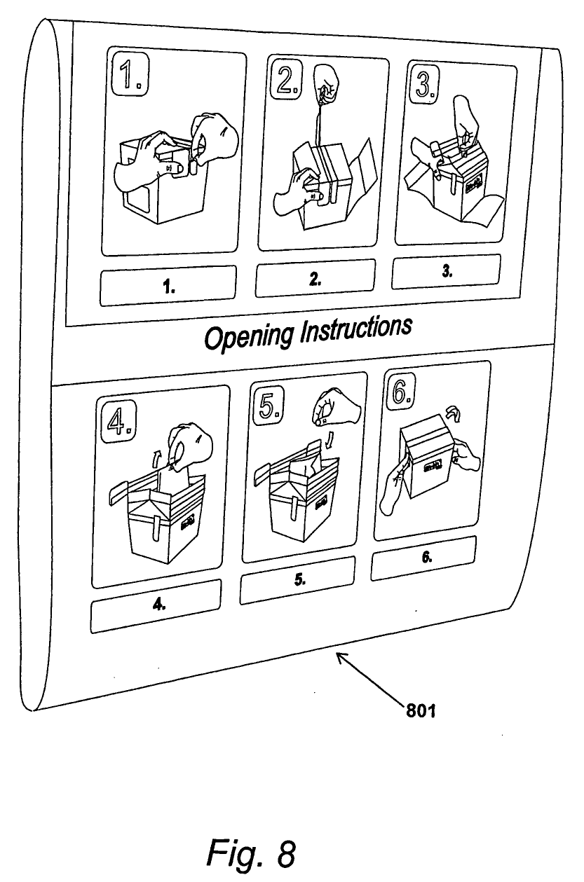

### FIG. 8

FIG. 8 shows a wraparound sleeve element 801 that is configured to extend around the container and over the closure portion of the container when the closure portion is secured in the second (closed) configuration. In an embodiment, the wraparound sleeve is fabricated from paper. In an arrangement, and as shown, the wraparound sleeve element may present text and/or graphics, such as information and/or instructions for use of the container.

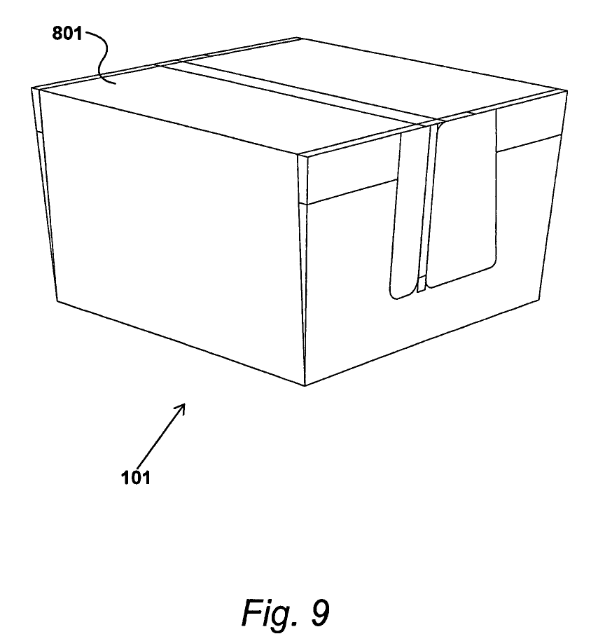

### FIG. 9

FIG. 9 shows container 101 in a closed configuration with wraparound sleeve 801 extending around the container. In the arrangement of FIG. 9, the second sealing element is in use to secure the closure portion of the container in the second (closed) configuration.

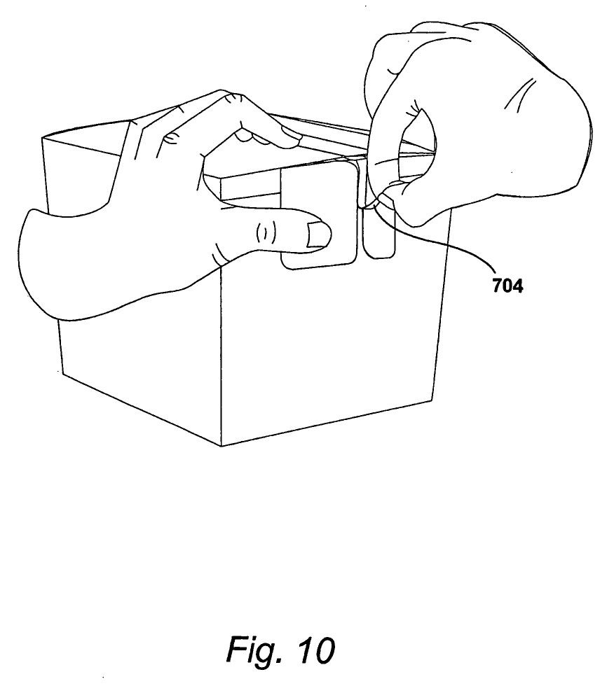

### FIG. 10

In FIG. 10, the container and wraparound sleeve element arrangement of FIG. 9 is received. To open the container the release element 704 is manually torn off as shown.

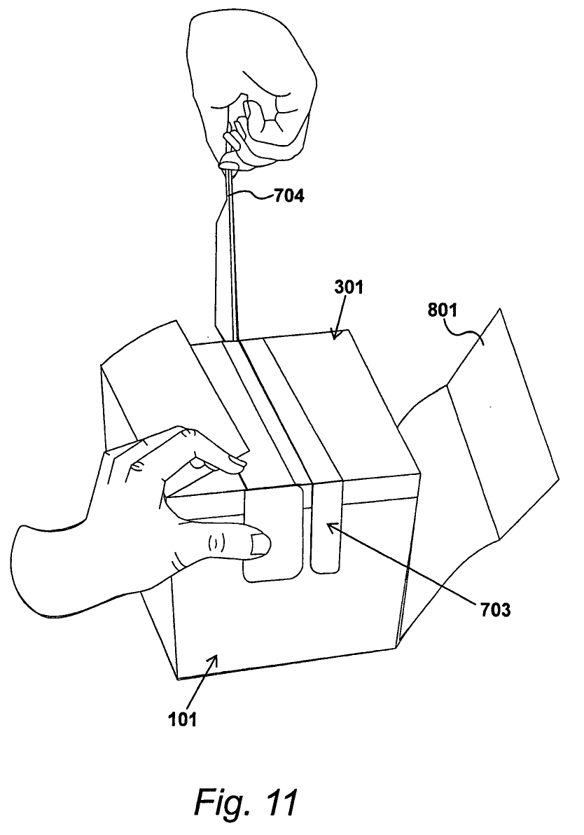

### FIG. 11

As the release element 704 is torn across the top surface 301 of the container, the wraparound sleeve element is broken apart. The second securing portion 703 remains secured to container 101.

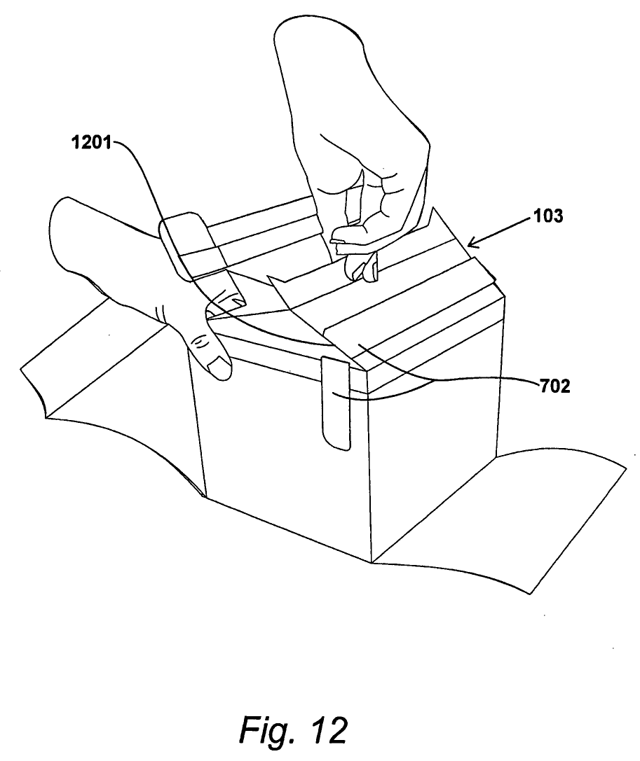

### FIG. 12

As shown in FIG. 12, once the release element has been fully torn off closure portion 103, and in turn container 101, may be manually opened at this stage, second securing portion 703 is also broken along perforations at point 1201 to enable the opening of container 101.

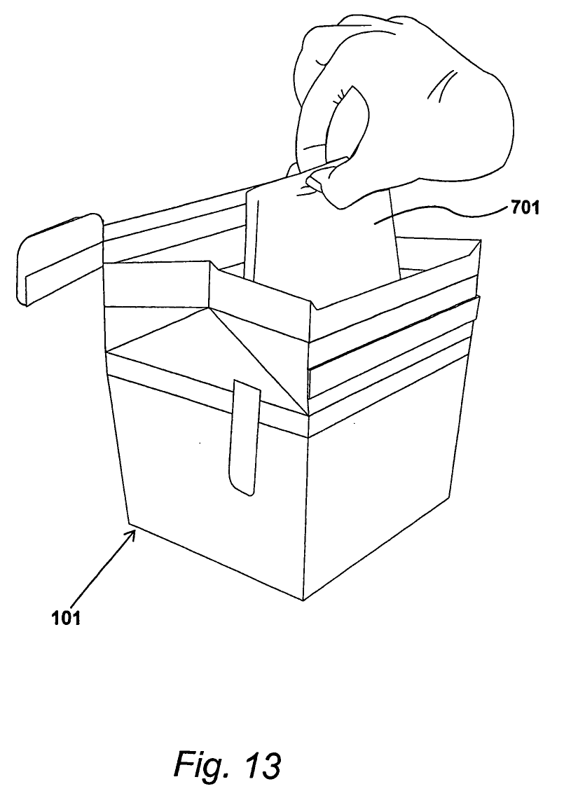

### FIG. 13

After the container 101 has been opened, following the procedure described with reference to FIGS. 10 to 12, it is then possible for items, such as item 701 to be removed, as illustrated in FIG. 13.

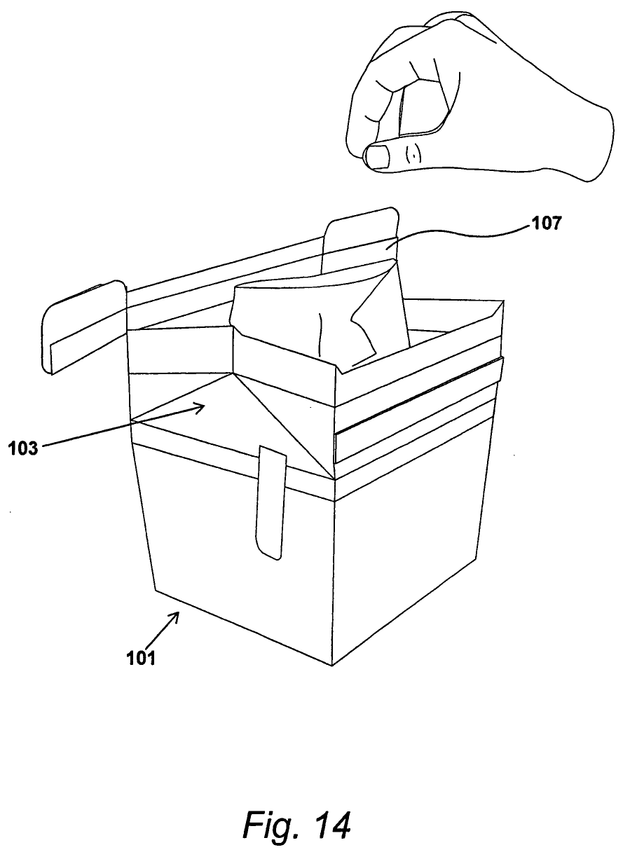

### FIG. 14

The container 101 may now be used to store items as desired. Such items may include tissues, pipettes, or other non-sharp items. Thus the container may be used to store items such as laboratory or medical implements.

Once the container is ready to be closed again, the closure portion 103 may be closed substantially as described with reference to FIGS. 2 to 5, but this time the container is sealed using first sealing element 107.

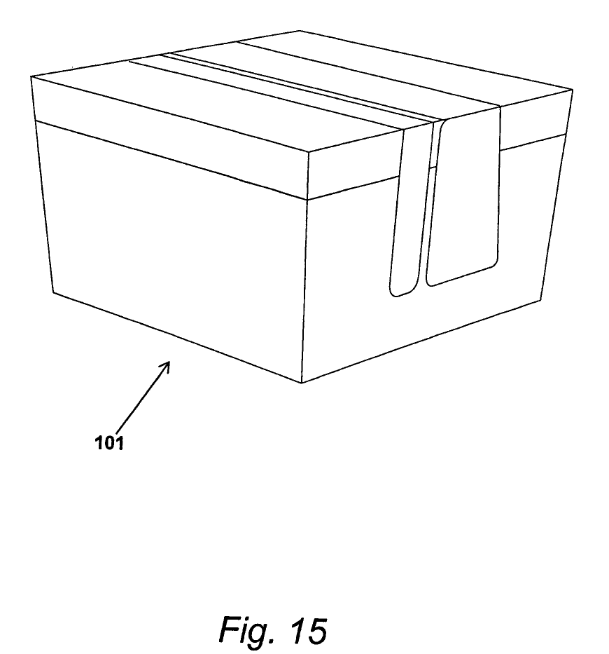

### FIG. 15

FIG. 15 shows container 101 having been resealed. In an embodiment, at least the securing element with which the container is resealed, it is configured to become unsecured in response to heat applied by the operation of an autoclave.

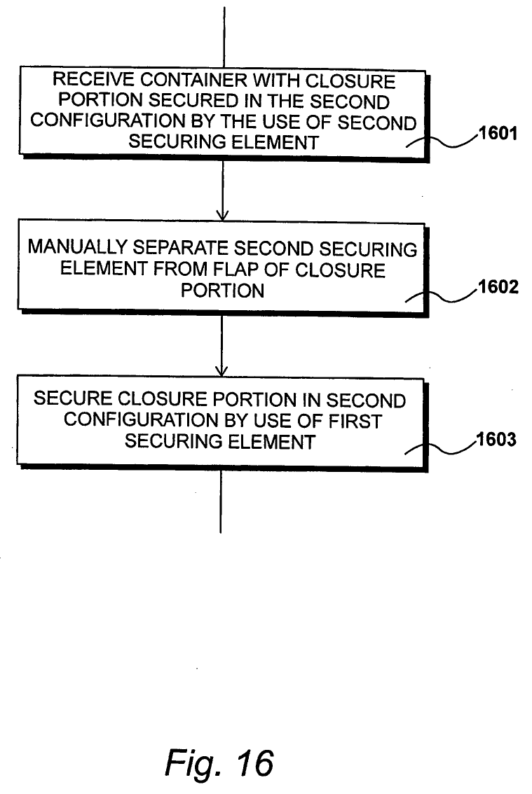

### FIG. 16

FIG. 16 shows steps in a method of use of the container. At step 1601 a container is received with the closure portion secured in the second configuration by use of the second securing element. At step 1602 an operation is performed to manually separate the second securing element from the flap of the closure portion. At step 1603 the closure portion is secured in the second configuration by use of the first securing element.

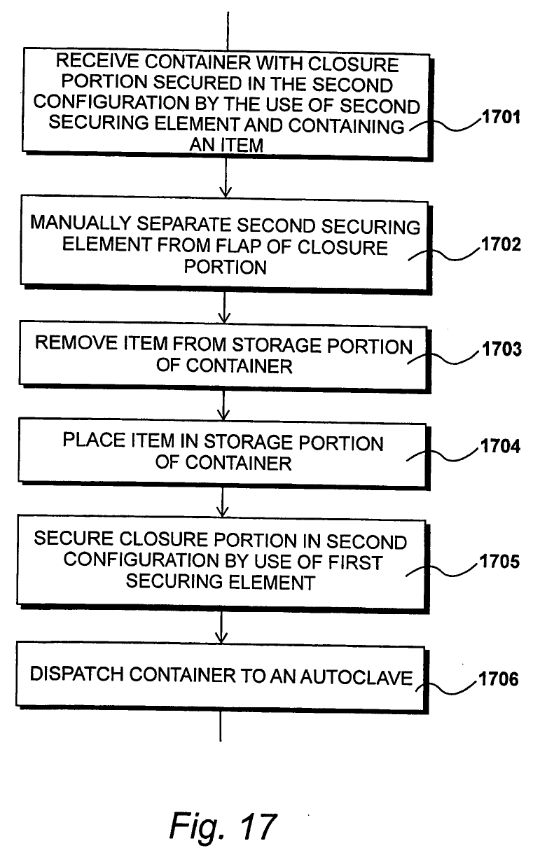

### FIG. 17

FIG. 17 illustrates steps of a further method of use of the container. At step 1701, the container is received with the closure portion secured in the second configuration by use of second securing element and containing an item. At step 1702, the second securing element is manually separated from the flap of the closure portion. A step 1703 an item is removed from the storage portion of the container. At step 1704 an item is placed in the storage portion of the container. At step 1705 the closure portion is secured in the second configuration by use of the first securing element. At step 1706 the sealed container is dispatched to an autoclave.

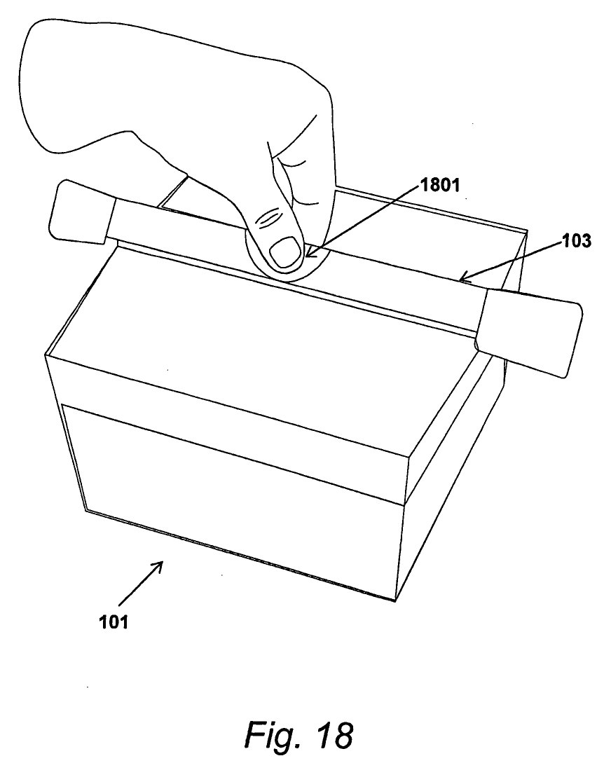

### FIG. 18

Container 101 may optionally be provided with a third temporary securing element 1801 configured to releasably maintain the closure portion 103 in the second (closed) configuration. In an embodiment, the third temporary securing element 1801 comprises a hook and loop fastening arrangement. Alternatively, any other suitable leads may be used, for example a bendable aluminum plate.

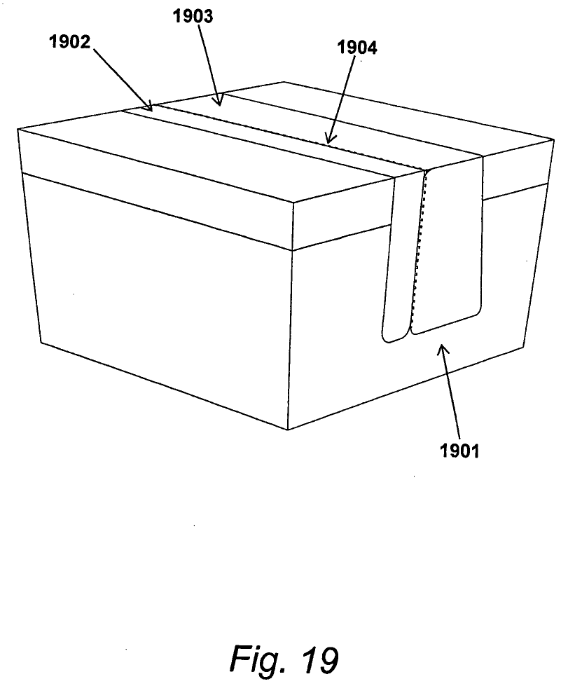

### FIG. 19

FIG. 19 shows a flap 1901 having an alternative arrangement to flap 105 as previously described. Flap 1901 comprises a first securing portion 1902, and a second securing portion 1903. Each of the securing portions 1902 and 1903 comprises a securing element, for example in a substantially similar manner to securing portion 702 and 703 of container 101. According to the arrangement of flap 1901, the flap comprises a row of perforations 1904 that is manually breakable to allow the first securing element to be manually separated from flap 1901.

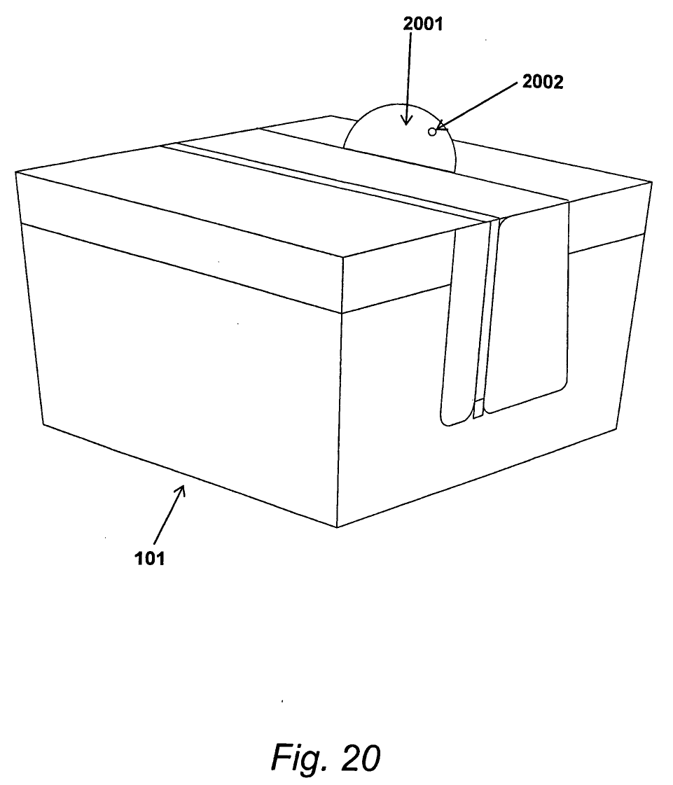

### FIG. 20

FIG. 20 shows a further optional feature of container 101. A handle element 2001 may be provided. The handle element 2001 may optionally provide an aperture 2002 to allow an identification tab to be attached to container 101. This is particularly useful when container 101 is being used to contain items that are contaminated with bodily fluids or other substances that may potentially present a hazard.

1. A container constructed substantially from card, comprising:
a storage portion having a base and defining an opening through which items are insertable into said storage portion, and
a closure portion foldable between a first configuration that allows access to said opening of said storage portion and a second configuration that closes said opening of said storage portion; and
said closure portion comprises a flap having a free edge, said flap comprising a securing portion presenting a first adhesive surface for securing said closure portion in said second configuration, said first securing portion being in the form of a strip extending along said flap; wherein
said flap further comprises a second securing portion having a second adhesive surface for securing said closure portion in said second configuration, said second securing portion being in the form of a strip attached to said first securing portion and extending alongside said first securing portion, and
one of said first securing portion and said second securing portion is manually separable from said flap.

2. The container of claim 1, wherein said one of said first securing element and said second securing element that is manually separable from said flap is proximate to said free edge of said flap.

3. The container of claim 1, wherein said flap comprises a row of perforations defining a fracture line that is manually breakable to allow said one of said first securing portion and said second securing portion to be manually separated from said flap.

4. (canceled)

5. The container of claim 1, wherein said flap comprises a release portion that is in the form of a tear off strip that extends along said flap between said first securing portion and second securing portion, and is manually separable from said flap.

6. (canceled)

7. The container of claim 1, further comprising a third temporary securing element configured to releasably maintain said closure portion in said second configuration.

8. The container of claim 7, wherein said third temporary securing element comprises a hook and loop fastening arrangement.

9. The container of claim 1, wherein the second adhesive surface of said second securing portion is configured to become unsecured in response to heat applied by the operation of an autoclave.

10. The container of claim 1, wherein said storage portion comprises side walls that taper outwardly from the base of said storage portion.

11. The container of claim 1, wherein said base has a substantially square shape.

12. The container of claim 1, wherein said card is coated with a plastics material.

13. The container of claim 1, provided with a wraparound sleeve element configured to extend around said container and over said closure portion when said closure portion is secured in said second configuration.

14. The container of claim 1, wherein said closure portion is configured to present a substantially planar surface when said closure portion is in said second configuration.

15. A method of use of the container of claim 1, said method comprising the steps of:
a) receiving said container with said closure portion secured in said second configuration by use of said second adhesive surface of said second securing portion,
b) manually separating said second securing portion from said flap, and
c) securing said closure portion in said second configuration by use of said first adhesive surface of said first securing portion.

16. The method of claim 15, wherein at step a) said container comprises an item placed in said storage portion and said method comprising the step of:
removing said item from said storage portion after step b) and before step c).

17. The method of claim 16, wherein said item is a laboratory or medical implement

18. The method of claim 15, wherein said container further comprises a third securing element configured to releasably maintain said closure portion in said second configuration, and said method further comprises the step of:
releasably maintaining said closure portion in said second configuration by use of said third securing element after step b) and before step c).

19. The method of claim 15, wherein said second securing element is configured to become unsecured in response to heat applied by the operation of an autoclave, and said method further comprises the step of:
dispatching said container to an autoclave after step c).

20. The method of claim 15, wherein at step a) said container is provided with a wraparound sleeve element that extends around said container and over said closure portion, and said method further comprises the step of:
removing said wraparound sleeve element from around said container after step a) and before step b).

21. A method of use of the container of claim 1, said method comprising the steps of:
inserting new unused medical equipment into said container and securing said container by said second securing element;
manually separating said second securing element from said flap;
removing said new medical equipment from said container, conducting a medical procedure and returning contaminated items to said container;
securing said container by said first securing element; and
disposing of said container with said contaminated items.

22. A method of claim 21, wherein said container further comprises a third securing element, and said method comprises the steps of:
inserting new unused medical equipment into said container and securing said container by said second securing element;
manually separating said second securing element from said flap;
removing said new medical equipment from said container, conducting a medical procedure and returning contaminated items to said container;
temporarily securing said container by said third securing element;
unsecuring said third securing element and inserting further contaminated items into said container;
securing said container by said first securing element; and
disposing of said container with said contaminated items.
